(19) United States
(12) Patent Application Publication
(10) Pub. No.: US 2026/0318305 A1
(43) Pub. Date: Oct. 15, 2026

(54) IMPELLER FOR AN IMPLANTABLE, VASCULAR SUPPORT SYSTEM

(71) Applicant: KARDION GMBH, Stuttgart (DE)

(72) Inventors: Armin Schuelke, Aidlingen (DE); Ingo Stotz, Ditzingen (DE); Johannes Bette, Leonberg (DE); David Minzenmay, Stuttgart (DE)

(21) Appl. No.: 19/431,644

(22) Filed: Dec. 23, 2025

Related U.S. Application Data
(63) Continuation of application No. 18/455,502, filed on Aug. 24, 2023, now Pat. No. 12,523,228.
Application No. 18/455,502 is a continuation of application No. 17/258,853, filed on Jul. 9, 2019, now Pat. No. 11,754,075.

(30) Foreign Application Priority Data
Jul. 10, 2018 (DE) 102018211327.0

(51) Int. Cl. F04D 13/02; A61M 60/174; A61M 60/237; A61M 60/408; A61M 60/416; A61M 60/419; A61M 60/804; A61M 60/82; A61M 60/857; F04D 3/02; F04D 29/18; F04D 29/20; F04D 29/60

(52) U.S. Cl. CPC F04D 13/024; A61M 60/174; A61M 60/237; A61M 60/408; A61M 60/416; A61M 60/419; A61M 60/804; A61M 60/82; A61M 60/857; F04D 3/02; F04D 29/181; F04D 29/20; F04D 29/60; A61M 2207/00

(57) ABSTRACT

The invention relates to an impeller (1) for an implantable, vascular support system (2), at least comprising: —an impeller body (3) having a first longitudinal portion (4) and a second longitudinal portion (5); —at least one blade (6) formed in the first longitudinal portion (4) and designed to axially convey a fluid by means of a rotational movement; —at least one magnet (7) provided and encapsulated in the second longitudinal portion (5).

### INCORPORATION BY REFERENCE TO ANY PRIORITY APPLICATIONS

The present application is a continuation of U.S. application Ser. No. 18/455502 entitled “IMPELLER FOR AN IMPLANTABLE, VASCULAR SUPPORT SYSTEM” filed Aug. 24, 2023, which is a continuation of U.S. application Ser. No. 17/258853 entitled “IMPELLER FOR AN IMPLANTABLE, VASCULAR SUPPORT SYSTEM” filed Jul. 27, 2021, which claims priority to PCT/EP 2019/068428 filed Jul. 9, 2019, which claims priority to DE 102018211327.0 filed Jul. 10, 2018. All of the above-listed applications and any and all other applications for which a foreign or domestic priority claim is identified in the Application Data Sheet as filed with the present application, are hereby incorporated by reference under 37 CFR 1.57, and form a part of this specification for all purposes.

### BACKGROUND

### Field

The invention relates to an impeller for an implantable vascular support system, an implantable vascular support system, and a method for producing an impeller for an implantable vascular support system. The invention is used in particular in (fully) implanted left ventricular assist devices (LVAD).

### Description of the Related Art

Today's LVAD cardiac support systems can generally be differentiated in terms of their position on the heart and their access to the blood stream. Approved long-term support systems are positioned at the apex of the heart (transapically) and bypass the left ventricle by pumping blood from the apex of the heart through a hose directly into the aorta (bypass). Since these systems are not located within the cardiovascular system itself, but are placed outside the heart, the available installation space for the pumps is relatively large. The housings in which the impeller moves are multiple centimeters in size.

A (percutaneous) minimally invasive or (fully) implanted support system in aortic valve position, which is currently used primarily for short-term support (so-called bridge to decision, bridge to transplant), represents another type of access. In this case, the natural aortic valve is used to create a separation between the pump inlet and the pump outlet. The advantage of this arrangement is that the aorta can be used as an access route (transfemoral or transaortic) in the context of a minimally invasive surgical procedure and there is no need for a sternotomy. With this type of support system, the installation space (length and outer diameter) is very limited due to the access route. A disadvantage of small pumps, however, is the comparatively low efficiency and the resulting reduced volume flow.

### SUMMARY

The object of the invention is to provide an impeller for an implantable vascular support system and an implantable vascular support system. One object of the invention is in particular to provide an impeller for an implantable vascular support system and a vascular support system which is (fully) implantable in aortic valve position, which is long-term stable and is suitable not only for short-term support of the heart function of a person and whereby the efficiency of the vascular support system is improved.

This object is achieved by the impeller described herein and the implantable vascular support system described herein, as well as the method for producing an impeller housing for an implantable vascular support system described herein.

Advantageous Embodiments Of The Invention Are Described Herein.

Proposed here is an impeller for an implantable vascular support system, at least comprising:
an impeller body having a first longitudinal portion and a second longitudinal portion,
at least one blade, which is formed in the first longitudinal portion and is configured to axially convey a fluid with a rotational movement,
at least one magnet, which is disposed and encapsulated in the second longitudinal portion.

In other words, the solution presented here in particular describes a preferably multipart impeller for a VAD pump having an integrated magnetic coupling. The solution presented here contributes advantageously to providing a long-term stable system for contactless torque transmission in an implantable vascular support system. The allocation of the functions fluid conveyance and torque transmission to different longitudinal portions of the impeller body makes it possible to achieve a particularly advantageous compact design with a comparatively small outer diameter and wall thicknesses, with which the limited installation space in VAD axial flow pumps, which are intended to be implanted inside the aorta, can be used to maximum advantage. In particular the encapsulation of the at least one magnet in the second longitudinal portion contributes to the long-term stability of the system. This advantageously allows the construction of a magnet system which can transmit the required torques and at the same time encapsulates the magnets in the motor and impeller in a biocompatible and long-term stable manner.

The impeller comprises an impeller body having a first longitudinal portion and a second longitudinal portion. In other words, this means in particular that the (elongated and/or hose-like) impeller body can be subdivided into a first longitudinal portion and a second longitudinal portion. In the case of a one-piece impeller body, this subdivision would be discernible only conceptually and/or by a difference in the wall thickness, for example.

In the case of a multipart impeller body, said impeller body could in particular be multipart in that the first longitudinal portion and the second longitudinal portion are separate parts or elements of the impeller body. In other words, in the case of a multipart impeller body, this means in particular that the first longitudinal portion describes a first impeller body element (which can also be described as a blading component) and the second longitudinal portion describes a second impeller body element (which can also be described as a magnet mount). In this case, the first impeller body element and the second impeller body element together form the impeller body. For this purpose, said elements are typically connected to one another (in a material-locking manner); for example, they can be (thermally) joined to one another at their ends, for example brazed or welded.

The impeller body can have the (basic) shape of a (elongated) cylinder, for example. “Elongated” here means in particular that the longitudinal extension is at least twice as large as the (maximum) diameter. The second longitudinal portion of the impeller body preferably has the shape of a (elongated) tube with a circular cross-section. The second impeller body element (if present) can have the shape of a pipe section.

The impeller further comprises at least one blade, which is formed in the first longitudinal portion and is configured to axially convey a fluid with a rotational movement. The blade can be formed in one piece with the first longitudinal portion, for example. The blade can alternatively be (detachably or fixedly) connected to the first longitudinal portion. The blade is preferably thermally joined to the first longitudinal portion. The blade is preferably configured to convey a fluid with a flow direction parallel to the longitudinal axis of the impeller. In other words, this means in particular that the impeller is configured to in particular act as an impeller of an axial flow pump. An axial flow pump is a pump that conveys fluids with a rotational movement axially, i.e. parallel to the axis of the pump shaft. For this purpose, the blade can be spatially curved.

The impeller further comprises at least one magnet, which is disposed in the second longitudinal portion and encapsulated (with respect to the environment). The (full) encapsulation of the at least one magnet advantageously contributes to the fact that the impeller can be used biocompatibly in the body, because the conveyed blood cannot come into direct contact with the magnet.

In order to transmit a torque radially, a plurality of magnet segments (usually between 4 and 8 magnet segments) having different magnetization directions are preferably used. These magnet segments are preferably disposed to form a pipe section or an annular arrangement of magnets. This pipe section or this arrangement of magnets preferably has a wall thickness of approx. 0.5 mm to 1.5 mm. A plurality of magnetic circuits can furthermore also be constructed in axial direction (e.g. a two-part magnetic coupling). Due to the high coercive field strength, the use of neodymium iron-boron magnets is preferred, but these are generally susceptible to corrosion and are therefore not readily biocompatible. For this reason, the encapsulation of such magnets is particularly advantageous.

The impeller body preferably comprises a magnet mount in the second longitudinal portion or the second longitudinal portion is shaped in the manner of a magnet mount. The magnet mount can be configured to accommodate a magnet assembly. In this context, the magnet assembly comprises at least one magnet and at least one magnetic return.

The magnetic return serves in particular to guide the magnetic field lines, which advantageously allows higher torques to be transmitted. The materials used are, for example, magnetizable, ferrous materials. The magnetic return is preferably configured in the form of a pipe section. This pipe section particularly preferably has a wall thickness of approx. 0.4 mm.

The magnet mount comprises a recess, for example, which can extend into the impeller body in radial direction (radial coupling) or in axial direction (axial coupling). This recess preferably has a rectangular cross-sectional shape.

The recess (the magnet mount) preferably extends radially inward from a jacket surface (cylinder outer surface) of the second longitudinal portion. In this context, it is further preferred for the recess to be delimited (in radial direction) by an inner wall of the impeller body, which in particular at the same time delimits (in radial direction) a space for accommodating an inner rotor or drive rotor that cooperates with the at least one magnet of the impeller.

The recess (of the magnet mount) further preferably extends (axially) from an end face of the second longitudinal portion of the impeller body facing away from the first longitudinal portion in the direction of the first longitudinal portion. In this context, it is further preferred for the recess to be delimited (in radial direction) by an inner surface of the impeller body, which is orthogonal to the longitudinal axis of the impeller body.

The recess is preferably configured and/or dimensioned and shaped such that it can fully accommodate the magnet assembly. In addition, the (only one) opening of the recess is preferably configured such that it can be closed (hermetically) with a cover. The recess and the cover particularly advantageously form a capsule, in which the magnet assembly or the at least one magnet is disposed and encapsulated.

In the second longitudinal portion, the impeller body preferably has a wall thickness that is less than or equal to 2 mm [millimeters], preferably less than or equal to 1.5 mm or even less than or equal to 1 mm. The wall thicknesses are particularly preferred in the range of 0.01 mm to 0.5 mm or even in the range of 0.05 mm to 0.2 mm. The wall with the correspondingly small wall thickness can be the above-described inner wall (e.g. in the case of a radial coupling), for example, or a jacket (e.g. in the case of an axial coupling) of the second longitudinal portion or a wall in the region of the magnet mount. The correspondingly small wall thickness can also pertain to the wall thickness of a cover or a sleeve for encapsulating the magnet. A correspondingly small wall thickness in the second longitudinal portion or in the region of the magnet mount contributes to this particularly advantageously, so that an advantageously high torque can be transmitted for cardiac support even in a small installation space.

According to one advantageous configuration, it is proposed that the impeller body be formed in one piece. In other words, this means in particular that the first longitudinal portion and the second longitudinal portion of the impeller body are formed in one piece (with one another). Preferably, in this context, the impeller body and the at least one blade are also formed in one piece (with one another). A milling process, a casting process or an additive manufacturing process, such as a 3D printing process, for example, can be used for one-piece production.

According to another advantageous configuration, it is proposed that the impeller body be formed in multiple parts. A multipart impeller body is particularly advantageous with respect to the mass moment of inertia of the rotor, the durability of the blades and/or the thrombogenicity of the surfaces. It can furthermore also be advantageous for manufacturing and assembly reasons as well as for cost reasons to construct the impeller in multiple parts.

A multipart impeller represents a particularly advantageous aspect of the solution presented here. This design advantageously allows a clear separation according to function, so that, for example, one part of the impeller is responsible for conveying the fluid (requirements: high degree of precision for the blade geometry, very smooth surfaces), another for transmitting the torque (requirements: hermetic tightness to ingress of fluid, high balancing quality). In the case of multipart production, individual parts/assemblies can particularly advantageously be tested separately prior to final assembly (e.g. for tightness due to the susceptibility of the magnets to corrosion, for functionality of the coupling or for rotational speed stability). Another advantage is the free combinability of the parts (the magnetic coupling remains the same, for example, but there are different blade geometries or materials, e.g. plastic blading for short-term use, titanium or ceramic blading for long-term use).

In this context, the first longitudinal portion and the second longitudinal portion are preferably thermally joined to one another. Said portions are preferably joined to one another at their ends. A welding process or a brazing process can be used for thermal joining. The first longitudinal portion and the second longitudinal portion are preferably connected to one another with a (radially) circumferential weld seam.

Furthermore, for a multipart design, long-term stable joints are particularly advantageous for long-term use and functional integrity. For this purpose, facing end faces of the first and second longitudinal portions can comprise centering elements which enable a thermal joining connection, such as a (radially) circumferential weld seam of particularly high quality.

According to one advantageous configuration, it is proposed that the second longitudinal portion forms a rotor for a magnetic coupling. The second longitudinal portion preferably forms an outer rotor for a magnetic coupling. A torque can thus be transmitted particularly advantageously without contact.

The second longitudinal portion preferably forms a first rotor of a magnetic coupling (which comprises two rotors). The second rotor can be formed with a drive shaft with which (drive) magnets are fixedly connected. The second longitudinal portion can thus represent an output side of a magnetic coupling. The magnetic coupling can be configured as a radial coupling or as an axial coupling. In the case of a radial coupling, the first rotor and the second rotor are disposed one behind the other in radial direction and at least partially overlapping in axial direction. In the case of an axial coupling, the first rotor and the second rotor are disposed one behind the other in axial direction and at least partially overlapping in radial direction.

The impeller or the impeller body is furthermore preferably made of a high-strength and/or biocompatible material. Grade 5 titanium, for example, can be used for this purpose. This results in the significant advantage that the support system can remain implanted for as long as possible. Grade 5 titanium can furthermore advantageously ensure a weldable joint.

According to a further aspect, an implantable vascular support system which comprises a here proposed impeller is proposed as well. The support system typically also comprises an impeller housing in which the impeller is held (in the manner of an impeller).

The vascular support system is preferably a cardiac support system, particularly preferably a ventricular support system. The support system is regularly used to support the conveyance of blood in the circulatory system of humans, e.g. a patient. The support system can be disposed at least partially in a blood vessel. The blood vessel is the aorta, for example, in particular in the case of a left heart support system, or the common trunk (truncus pulmonalis) into the two pulmonary arteries, in particular in the case of a right heart support system. The support system can preferably be disposed at the outlet of the left ventricle of the heart or the left ventricle. The support system can particularly preferably be disposed in aortic valve position.

The support system is preferably a left ventricular cardiac support system (LVAD) or a percutaneous, minimally invasive left heart support system. The system is furthermore preferably fully implantable. In other words, this means in particular that the means required for conveying blood, in particular a flow machine (having an impeller and an impeller housing) of the support system, are located entirely within the patient's body (in particular in the heart and/or the aorta) and remain there. However, it is not mandatory that a control device or an evaluation device of the support system also be disposed inside the patient's body. For example, the support system can be implanted such that the control device or evaluation device is disposed on the patient's skin or outside the patient's body and a connection to the flow machine disposed inside the body is established. The support system is particularly preferably configured and/or suited to being disposed at least partially in a ventricle, preferably in the left ventricle of a heart, and/or in an aorta, in particular in aortic valve position.

The support system further preferably comprises a cannula, in particular an inlet cannula, a flow machine, such as a pump (with an impeller), and/or an electric motor. The electric motor is regularly a component of the flow machine. The (inlet) cannula (suction hose) is preferably configured such that, in the implanted state, it can conduct fluid from a (left) ventricle of a heart to the flow machine. The support system is preferably elongated and/or hose-like. The cannula and the flow machine are preferably disposed in the region of opposite ends of the support system.

The support system is in particular configured such that it can be implanted (in a minimally invasive manner) in aortic valve position. For this purpose, the support system preferably has an outer diameter in the range of 4 mm to 10 mm, particularly preferably in the range of 5 mm to 8 mm and/or a (rigid) length in the range of 10 mm to 80 mm, particularly preferably in the range of 20 mm to 50 mm. Such a dimensioning of the support system advantageously allows the support system to be implanted in a minimally invasive manner in aortic valve position while still maintaining good functionality, in particular comparatively high efficiency. In the case of a transfemoral access route, the installation space for the support system (the pump) is very limited because the femoral artery has a relatively small diameter, so the outer diameter of the support system should be as small as possible. The efficiency of an impeller of the pump should nonetheless not become too low. The support system is furthermore pushed around the aortic arch, which can typically describe an arc of >180 °. To overcome this anatomical constriction, the overall rigid length of an implant should also be kept as small as possible.

According to a further aspect, a method for producing an impeller for an implantable, vascular support system is proposed as well and at least comprises the following steps:
a) providing an impeller body having a first longitudinal portion and a second longitudinal portion, wherein at least one blade is formed in the first longitudinal portion and configured to axially convey a fluid by means of a rotational movement,
b) providing at least one magnet,
c) disposing and encapsulating the magnet in the second longitudinal portion.

The specified sequence of the method steps a), b) and c) is only an example and can be the result of a regular operating sequence.

According to one advantageous configuration, it is proposed that the impeller body be provided in one piece in Step a). In this context, it is advantageous if the at least one magnet or the one magnet assembly is (initially) disposed in a sleeve and/or (fixedly) connected to said sleeve. The at least one magnet or the magnet assembly can furthermore (then) be pushed onto the second longitudinal portion of the impeller body in and/or with the sleeve. In this context, it is particularly advantageous if the sleeve is configured or shaped such that, in the pushed-on state, it encapsulates the at least one magnet or the magnet assembly in cooperation with the second longitudinal portion. It is furthermore advantageous if the joints between the sleeve and the impeller body (radial and axial) are thermally joined, in particular welded.

During operation, the sleeve serves in particular to encapsulate the non-biocompatible materials (magnets, return). In order to take up as little installation space as possible, it is particularly advantageous if the wall thickness of the sleeve is kept as low as possible. In this context, a preferred wall thickness of the sleeve is approx. 0.1 mm. In particular if the sleeve is to be machined, a biocompatible metal can be used, e. g. Grade 5 titanium. The material in the region between the two magnetic rotors can furthermore also be non-magnetic, e. g. titanium or plastic. This material can be formed in the manner of a so-called split case of a magnetic coupling, for example. This in particular does not (disadvantageously) affect the function of the magnetic coupling.

The joints between the sleeve and the impeller body are advantageously welded hermetically tight. The mentioned assembly steps (pushing on the sleeve with the magnets located therein) result in particular in a radial and an axial weld seam. The magnet system in the impeller is thus advantageously completely encapsulated, but can nonetheless transmit the magnetic forces and with it the torque in a particularly advantageous manner without contact.

According to another advantageous configuration, it is proposed that the impeller body be provided in multiple parts in Step a). A separation (between the first longitudinal portion and the second longitudinal portion) advantageously results in further degrees of freedom also in terms of production. The blading can also be produced in an investment casting process, for example, or via 3D printing. In particular when using hard materials (ceramic or DLC layers), the bearing function can be implemented in the geometry. All in all, a more cost-effective and significantly more flexible production can advantageously be achieved.

In this context (multipart impeller body), the second longitudinal portion is preferably assembled before the second longitudinal portion is connected to the first longitudinal portion. This assembly can, for example, include an insertion of the at least one magnet or a magnet assembly into a magnet mount. This assembly can furthermore (subsequently) include closing the magnet mount with a cover and, if necessary, thermally joining the cover to the second longitudinal portion.

Further preferably, in this context (multipart impeller body), a connection, in particular thermal joining of the first longitudinal portion and the second longitudinal portion, is carried out. It is furthermore preferred that a testing or inspection of the first longitudinal portion and/or the second longitudinal portion (independent of one another), in particular of the joints of the second longitudinal portion, take place (chronologically) before the connection (and after the above-described assembly of the second longitudinal portion). This can include a tightness test of the second longitudinal portion, in particular the magnet mount of the second longitudinal portion. This can alternatively or cumulatively include a test of the second longitudinal portion for tightness of the joints (because of the susceptibility of the magnets to corrosion), for functionality of the coupling and/or for rotational speed stability.

According to one advantageous configuration, it is proposed that a magnet assembly comprising the at least one magnet and a magnetic return be provided in Step b). The magnetic return serves in particular to guide the magnetic field lines, which advantageously allows higher torques to be transmitted. The magnetic return is in particular disposed such that it surrounds the at least one magnet circumferentially.

According to one advantageous configuration, it is proposed that the magnet be encapsulated with a cover in Step c). The cover can be a sleeve, for example, which is configured such that the at least one magnet or the magnet assembly can be pushed onto the second longitudinal portion of the impeller body in and/or with the sleeve. The cover can furthermore be a cover that is configured to close an (radial or axial) opening of a magnet mount. In this context, the cover can be formed with a (thin-walled) pipe section or a (thin-walled) wound (and possibly longitudinally welded) sheet metal, for example.

The details, features and advantageous configurations discussed in connection with the impeller can correspondingly also occur in the support system and/or the method presented here and vice versa. In this respect, reference is made in full to the statements there for a more detailed characterization of the features.

### BRIEF DESCRIPTION OF THE DRAWINGS

The solution presented here as well as its technical environment are explained in more detail below with reference to the figures. It is important to note that the invention is not intended to be limited by the design examples shown. In particular, unless explicitly stated otherwise, it is also possible to extract partial aspects of the facts explained in the figures and to combine them with other components and/or insights from other figures and/or the present description. The figures show schematically:

FIG. 1: a here proposed impeller in an impeller housing,

FIG. 2: another here proposed impeller in an impeller housing,

FIG. 3: a here proposed impeller,

FIG. 4: a sectional view of another here proposed impeller,

FIG. 5: a sequence of a here proposed method,

FIG. 6: a sequence of a further here proposed method,

FIG. 7: a sequence of a further here proposed method,

FIG. 8: an illustration of a further here proposed method, and

FIG. 9: a support system, implanted in a heart.

### DETAILED DESCRIPTION

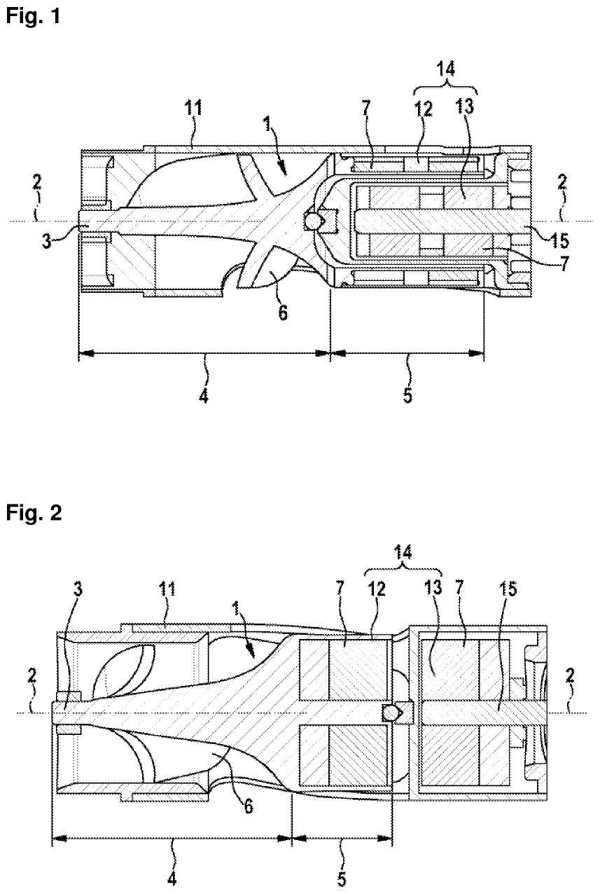

FIG. 1 schematically shows a here proposed impeller 1 in an impeller housing 11. The impeller 1 is suitable for an implantable vascular support system (not shown here, see FIG. 9). The impeller 1 can generally also be used in small axial flow pumps (with impeller), in particular with contactless torque transmission.

The impeller 1 comprises an impeller body 3 which is rotatable about an axis of rotation 2 and has a first longitudinal portion 4 which extends in the direction of the axis of rotation 2 and a second longitudinal portion 5 which extends in the direction of the axis of rotation 2. The impeller 1 further comprises at least one blade 6, which is formed in the first longitudinal portion 4 and is configured to axially convey a fluid in the direction of the axis of rotation 2 with a rotational movement. The impeller 1 also comprises at least one magnet 7, which is disposed and encapsulated in the second longitudinal portion 5.

In FIG. 1, the impeller body 3 is formed in one piece. For this purpose, the first longitudinal portion 4 and the second longitudinal portion 5 of the impeller body 3 are formed in one piece.

FIG. 1 further illustrates that the second longitudinal portion 4 forms a first rotor 12 (outer rotor) for a magnetic coupling 14. The first rotor 12 cooperates (for radial torque transmission) with a second rotor 13 (inner rotor). The second rotor 13 is formed with magnets 7, which are fixedly connected to a drive shaft 15. The first rotor 12 and the second rotor 13 form the magnetic coupling 14. In the embodiment according to FIG. 1, the magnetic coupling 14 is formed in the manner of a radial coupling system.

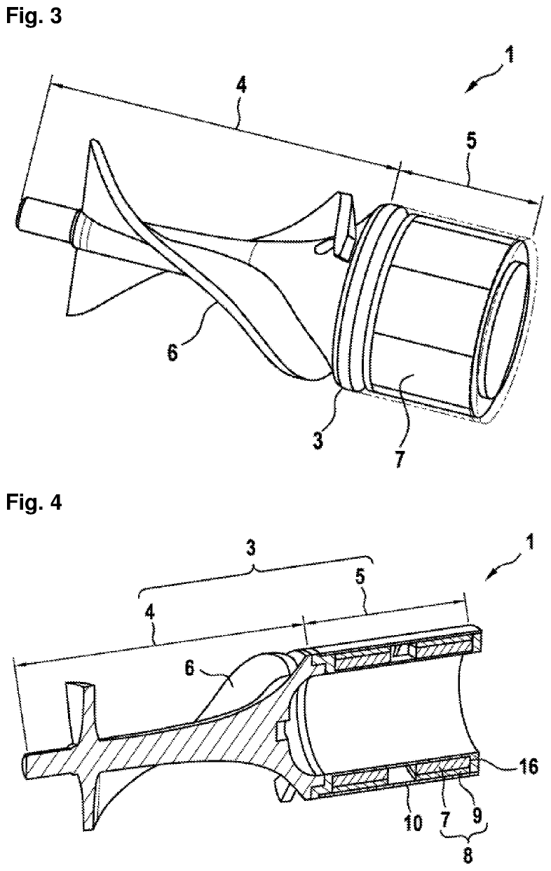

FIG. 2 schematically shows another here proposed impeller 1 in an impeller housing 11. The reference signs are used consistently, so that reference can be made in full to the statements regarding FIG. 1.

The impeller body 3 in FIG. 2 is likewise formed in one piece. The design variant according to FIG. 2 differs from the design variant according to FIG. 1 in particular in that the magnetic coupling 14 in FIG. 2 is formed in the manner of an axial coupling system. For this purpose, the first rotor 12 and the second rotor 13 cooperate accordingly (for axial torque transmission).

FIG. 1 and FIG. 2 show two fundamental options for transmitting the torque from the motor shaft 15 to the impeller 1 without contact using different magnet system arrangements (radial, axial). The transmittable torque depends in particular on one or more of the following factors:
The larger the magnets, the higher the transmittable torque. The magnet size is limited by the available installation space.
The smaller the distance between the magnet systems (motor shaft and impeller), the higher the transmittable torque. The distance results in particular from the wall thickness of the encapsulation and the various gap dimensions.
Arrangement and/or number of magnetic poles.
Material characteristics, such as energy density, remanence, coercive field strength and/or saturation polarization.

FIG. 3 schematically shows a here proposed impeller 1. The reference signs are used consistently, so that reference can be made in full to the statements regarding the preceding figures.

FIG. 3 shows an example of a finally assembled impeller 1 for an 8-pole radial coupling in a perspective view. The impeller body 3 in FIG. 3 is likewise formed in one piece.

FIG. 4 schematically shows another here proposed impeller 1. The reference signs are used consistently, so that reference can be made in full to the statements regarding the preceding figures.

In FIG. 4, the impeller body 3 is formed in multiple parts. For this purpose, the first longitudinal portion 4 and the second longitudinal portion 5 of the impeller body 3 are initially provided as separate or discrete components and then (fixedly) connected to one another to form the impeller body 3.

According to the illustration according to FIG. 4, the second longitudinal portion 5 forms a magnet mount 16, in which the at least one magnet 7 is disposed and encapsulated. As an example, the magnet 7 here is a component of a magnet assembly 8, which comprises the magnet 7 and a magnetic return 9. A cover 10, which (hermetically) closes the magnet mount 16, contributes to the encapsulation.

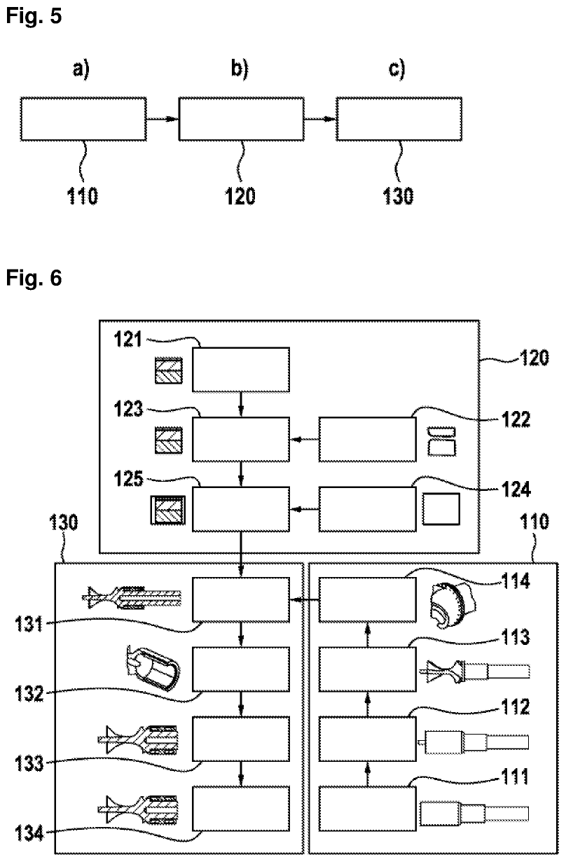

FIG. 5 schematically shows a sequence of a here proposed method. The method is used to produce an impeller for an implantable vascular support system. The shown sequence of the method steps a), b) and c) with blocks 110, 120 and 130 is only an example and can be the result of a regular operating sequence. In Block 110, an impeller body is provided, which has a first longitudinal portion and a second longitudinal portion and wherein at least one blade is formed in the first longitudinal portion and configured to axially convey a fluid by means of a rotational movement. In Block 120, at least one magnet is provided. In Block 130, the magnet is disposed and encapsulated in the second longitudinal portion.

FIG. 6 schematically shows a sequence of a further here proposed method. The method according to FIG. 6 is based on the sequence shown in FIG. 5, wherein examples of the configurations of the method steps a), b) and c) with blocks 110, 120 and 130 are explained in more detail. The method according to FIG. 6 is used to produce an impeller 1 with a one-piece impeller body 3, which can be driven with radial (contactless) torque transmission.

In Block 110, an impeller body is provided with a first longitudinal portion and a second longitudinal portion, wherein at least one blade is formed in the first longitudinal portion and configured to convey a fluid axially with a rotational movement. In other words, it can also be said that, in Block 110, an impeller assembly is provided.

For this purpose, a base body is turned and, if necessary, ground in a Block 111. The impeller or the impeller body is subsequently rough turned in a Block 112. Then, in a Block 113, the impeller or the impeller body, in particular the at least one blade, is milled. Subsequently, flushing bores are drilled as an example here in a Block 114. The flushing bores establish a connection between the main blood flow outside and the blood gap inside the impeller and contribute to a continuous exchange of the blood in the gap geometries, in order to prevent thrombus formation and the occurrence of blood damage mechanisms. The bore diameters are advantageously between 0.2 and 0.8 mm. This is an example that, as in Step a), the impeller body can be provided in one piece.

In Block 120, at least one magnet is provided. In other words, it can also be said that, in Block 120, a magnet system assembly is provided.

For this purpose, the magnets are segmented and magnetized (possibly, even ahead of time) in a Block 121. Furthermore, in a Block 122, a magnetic return is turned and ground. The magnets and the magnetic return are then glued in a Block 123. A sleeve (cover) is subsequently turned in a Block 124. The magnet system is then joined to the sleeve (e.g. by gluing and/or press-fitting) in a Block 125. This is an example that, as in Step b), a magnet assembly can be provided, which comprises the at least one magnet and a magnetic return. In the case of a system without a magnetic return, Blocks 122 and 123 can be omitted.

In Block 130, the magnet is disposed and encapsulated in the second longitudinal portion. In other words, it can also be said that, in Block 130, an overall system assembly is provided.

For this purpose, the magnetic system (from Block 120) is joined to the impeller or the impeller body (from Block 110) in a Block 131. The magnet or the magnet system is encapsulated with the cover (sleeve). The joints are subsequently welded tight (radially and axially) in a Block 132. The clamping spigot is then removed in a Block 133. The clamping spigot is kept in place until Block 133 for handling reasons.

The inner geometry of the impeller body is then turned out in a Block 134. To hollow out the inner geometry by machining, the clamping spigot is first removed (Block 133). The entire system is now held on the welded sleeve. Since the assembly is already assembled, the inner wall thickness (made here of titanium, for example) can also be very thin (wall thickness here approx. 0.1 mm, for example).

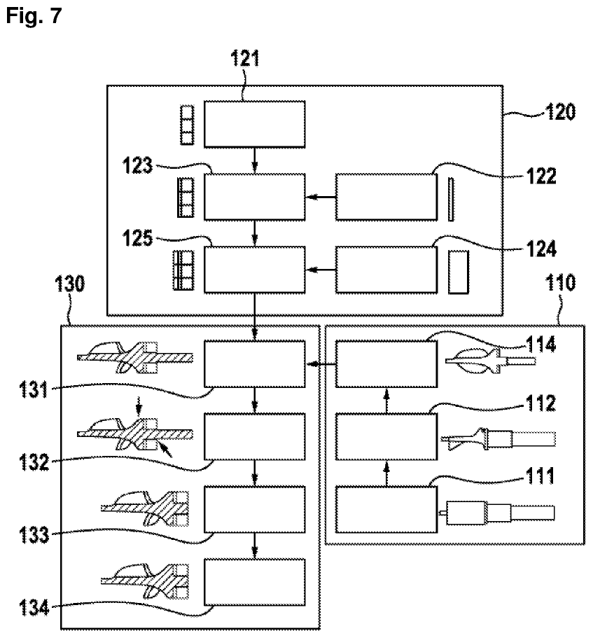

FIG. 7 schematically shows a sequence of a further here proposed method. The method according to FIG. 7 is based on the sequence shown in FIG. 5, wherein examples of the configurations of the method steps a), b) and c) with blocks 110, 120 and 130 are explained in more detail. The method according to FIG. 7 is used to produce an impeller 1 with a one-piece impeller body 3, which can be driven with axial (contactless) torque transmission.

In Block 110, an impeller body is provided with a first longitudinal portion and a second longitudinal portion, wherein at least one blade is formed in the first longitudinal portion and configured to convey a fluid axially with a rotational movement. In other words, it can also be said that, in Block 110, an impeller assembly is provided.

For this purpose, the impeller or the impeller body is rough turned in a Block 111. Then, in a Block 112, the impeller or the impeller body, in particular the at least one blade is milled and flushing bores are provided as an example. The flushing bores establish a connection between the main blood flow outside and the blood gap inside the impeller and contribute to a continuous exchange of the blood in the gap geometries, in order to prevent thrombus formation and the occurrence of blood damage mechanisms. The bore diameters are advantageously between 0.2 and 0.8 mm. The impeller body, in particular the second longitudinal portion of the impeller body, is then turned to a magnet diameter in a Block 114. This is an example of how the impeller body can be provided in one piece in Step a).

In Block 120, at least one magnet is provided. In other words, it can also be said that, in Block 120, a magnet system assembly is provided.

For this purpose, the magnets are segmented and magnetized in a Block 121 (or even earlier). Furthermore, in a Block 122, a magnetic return is turned. The magnets and the magnetic return are then glued, for example, in a Block 123. A sleeve (cover) is subsequently turned in a Block 124. The magnet system is then glued to the sleeve in a Block 125. This is an example of how a magnet assembly comprising the at least one magnet and a magnetic return can be provided in Step b). In the case of a system without a magnetic return, Blocks 122 and 123 can be omitted.

In Block 130, the magnet is disposed and encapsulated in the second longitudinal portion. In other words, it can also be said that, in Block 130, an overall system assembly is provided.

For this purpose, the magnetic system (from Block 120) is joined to the impeller or the impeller body (from Block 110) in a Block 131. The magnet or the magnet system is encapsulated with the cover (sleeve). The joints are subsequently welded tight (radially and axially) in a Block 132. The clamping spigot is then removed in a Block 133. The clamping spigot is kept in place until Block 133 for handling reasons.

The inner geometry of the impeller body is then turned out in a Block 134. To hollow out the inner geometry by machining, the clamping spigot is first removed (Block 133). The entire system is now held on the welded sleeve. Since the assembly is already assembled, the inner wall thickness (made here of titanium, for example) can also be very thin (wall thickness here approx. 0.1 mm, for example).

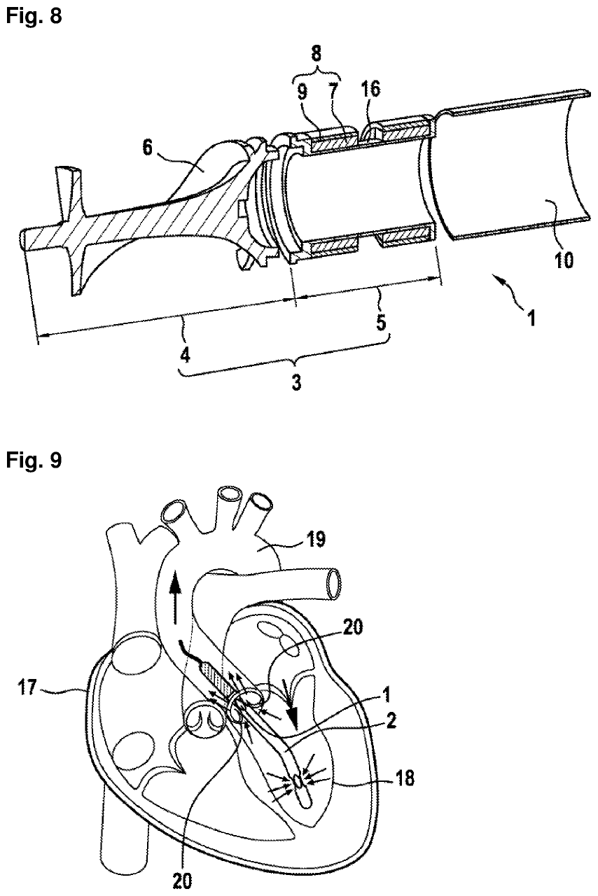

FIG. 8 schematically shows an illustration of a further here proposed method. The reference signs are used consistently, so that reference can be made in full to the statements regarding the preceding figures (in particular FIGS. 1, 2, 3 and 4).

The design variant according to FIG. 8 is an example of how the impeller body 3 can be provided in multiple parts in Step a). In the course of assembly, the magnets 7 can first be joined to the magnet mount 16 (e.g. by gluing). The cover 10 is then pushed on and welded tight. The magnet 7 or the magnet assembly 8 is encapsulated with the cover 10. Finally, the blading 6 is mounted and also welded.

When using ceramics, it is particularly advantageous to apply a metallization in advance in order to be able to connect the parts by means of welding or laser brazing. Glued connections are possible as well, since the connection between the blading 6 and the magnet 7 does not have to be tight.

The multipart nature of the impeller body 3 can be seen clearly in the exploded view of FIG. 8. The cover 10 can be made from a thin-walled tube or wound from a thin sheet metal and welded longitudinally.

FIG. 9 schematically shows a support system 2 implanted in a heart 17. The reference signs are used consistently, so that reference can be made in full to the statements regarding the preceding figures.

FIG. 9 shows a ventricular support system 2, i.e. the support system 2, projecting into a (here left) ventricle 18 of the heart 17. The support system 2 is furthermore disposed in aortic valve position, i.e. the support system 2 intersects a cross-section in which the aortic valves 20 are located. The support system 2 supports the heart 17 by conveying or pumping blood from the ventricle 18 into the aorta 19. The blood flow is indicated in FIG. 9 with arrows.

The support system 2 comprises an impeller 1 (in the manner of an impeller), which is surrounded by a (here not depicted) impeller housing. In the example of an alignment of the support system 2 shown in FIG. 9, the impeller 1 is located in the aorta 19.

1.-10. (canceled)

11. An impeller for an implantable vascular support system, comprising:
an impeller body having a first longitudinal portion and a second longitudinal portion;
at least one blade formed in the first longitudinal portion and is configured to axially convey a fluid by means of a rotational movement; and
at least one magnet disposed and encapsulated in the second longitudinal portion.

12. A method for producing an impeller for an implantable vascular support system, comprising:
providing an impeller body having a first longitudinal portion and a second longitudinal portion, wherein at least one blade is formed in the first longitudinal portion and configured to axially convey a fluid by means of a rotational movement;
providing at least one magnet;
disposing and encapsulating the magnet in the second longitudinal portion.
